(19) United States
(12) Patent Application Publication
(10) Pub. No.: US 2008/0294038 A1
(43) Pub. Date: Nov. 27, 2008

(54) Model-Based Flow Analysis and Visualization

(75) Inventors: Juergen Weese, Aachen (DE); Alexandra Groth, Aachen (DE); Joerg Bredno, Aachen (DE); Tom Bruijns, Best (NL); Peter Rongen, Eindhoven (NL); Roel Hermans, Den Bosch (NL); Heidrun Steinhauser, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/096,436

(22) PCT Filed: Nov. 15, 2006
(86) PCT No.: PCT/IB2006/054279

§ 371 Date: Jun. 6, 2008

Related U.S. Application Data
(60) Provisional application No. 60/748,808, filed on Dec. 9, 2005.

(51) Int. Cl. A61B 6/00; G06G 7/60

(52) U.S. Cl. 600/431; 703/11; 382/128

(57) ABSTRACT

A system (900), method (100, 200) and apparatus (600, 700, 800) are provided for analyzing a blood flow in a vascular system from a dynamic diagnostic observation sequence (101) to determine blood flow parameters (112) for further determination of filters, replay speed and finally visualization of the replayed original and filtered sequences. A first embodiment (100) extracts features of the observation and uses these features to select an appropriate model from a database of pre-determined models of vascular system of interest which have associated parameters. These parameters are varied to create an instance of the model that best matches the original observation. A second embodiment (200) visualizes a replay of the original observation (101) and the observation (101′) predicted by the model to highlight differences therebetween. A third embodiment (800) provides filtering and control of the replay speed.

The present invention relates to a system, apparatus and method for deriving models of blood flow in vessels based on a sequence of images matching the derived models with standard blood vessel models to automatically measure properties of blood flow, identify anomalies, and visualize the results for further consideration by a physician or interventionalist by exploiting the model.

Many medical imaging modalities provide information to physicians and interventionalists concerning blood flow in different vascular systems. Automated and computer-aided analysis of clinical observations has been one focus of research and development for more than a decade. This also holds for flow analysis of angiographic acquisitions. The main objective of such an analysis is the robust extraction of quantitative and characteristic flow properties from a sequence of observed images showing the dynamics of a contrast agent in the blood stream.

Such an analysis has to deal with fluid properties of blood, the heartbeat, image noise, the contrast agent injection, and other properties that cannot be fixed in clinical acquisitions or are patient-specific. Therefore, an important property of any automated flow analysis is that it be able to deal with all known influences that determine the appearance of features. However, this a-priori knowledge of such a large set of different influences is difficult to incorporate into an analysis based on the interpretation of observed features, therefore leaving most currently known methods insufficiently robust for clinical usage.

The extraction of functional information from diagnostic acquisitions of the vascular system that image the advance of contrast agent through a vessel subsystem can provide a primary measurement of these influences. For example, for stenosis grading, the pressure decrease over the stenosis is of major interest to the treating physician. For aneurysm grading, the amount of blood that passes by the aneurysm without taking a detour through the aneurysm might be of interest, whereas for a bifurcation the fraction of flow into the branches is important functional information. The case at hand dictates what functional information is relevant. All known algorithms for quantitative blood flow assessment are based on a simple feature analysis such as the arrival time of a bolus of injected contract material and are unspecific as well as insufficient for the assessment of complex vessel configuration.

Blood flow measurements are essential for assessing the severity of diseases in arteries or veins (e.g. stenoses or aneurysms). The advance of contrast agent can be imaged by interventional x-ray, Ultrasound, repeated acquisitions using computed tomography or magnetic resonance imaging and other modalities. Examples are given for interventional x-ray; however, this is by way of example only and does not imply any limitation to x-ray modalities.

In a minimally-invasive procedure, an interventionalist inserts a catheter into the vessels of interest and injects a contrast agent to make the blood flow visible in an x-ray sequence thereof. Subsequently, the physician can assess the blood flow by a visual inspection of the spreading of the contrast agent in an acquired x-ray sequence. For the optimal visual impression of the fluid dynamics in the x-ray sequence, image pre-processing is required. For example, the removal of background noise is essential since it results in unsatisfactory visual impression. This applies, in particular, to flow sequences acquired at high frame rates because low image quality is obtained due to the low frame dose that has to be used in order to keep the overall patient dose expectable. One common noise suppression method is temporal filtering in which a given number of frames are weighted and averaged.

Up to now, signal processing has been performed with a fixed parameter set without accounting for the patient's individual blood flow. As a result, the visual impression of the fluid dynamic effects can be disturbed by inappropriate parameters. In the case of temporal filtering, the strength of temporal filtering is crucial. If the filtering strength is chosen too high, the bolus of contrast agent radically changes its position during imaging. As a result, a blurred bolus is displayed and important functional information is lost. Hence, the strength of temporal filtering has to be adapted to the actual flow speed, which is highly patient-, disease-, and organ-dependent. Furthermore, contrast agent mainly arrives in a bolus of high concentration and the visualization of observations is often tuned to show this bolus arrival whereas much diagnostically relevant information is contained in microflow phenomena which manifest in local, smaller variations of contrast agent concentration. These are often obscured by the major contrast agent bolus and methods to reveal and visualize microflow phenomena are desired.

Functional information allows a direct measurement of the impact of a disease on the human body and while not normally available is highly desirable. Thus, there is a growing demand for the extraction of functional information from medical imaging. However, blood flow analysis is not clinically routine because the information that can be automatically obtained from contrasted x-ray images or other modalities is not yet sufficient.

The system, apparatus and method of the present invention provide specific flow analysis based functional information concerning the underlying physical blood flow of an individual, i.e., parameters of the blood flow of a specific patient in an imaged vascular subsystem of interest. The flexible incorporation of a-priori knowledge into the blood flow analysis of the system, apparatus and method of the present invention is a paradigm shift from the prior art computational analysis of features to a new model-based functional analysis based on suitably selected prediction models.

In a first embodiment, a priori knowledge is derived from fluid dynamics and is complemented by available patient-specific information obtained from a sequence of one or more blood flow images, wherein the images are used to adapt a suitably selected model of the behavior of blood flow to the real physiological process represented by the sequence of patient blood flow images. As a basic advantage of the present invention, it is no longer necessary to formulate and implement feature analysis algorithms to explain all possible deviations of an observation (sequence of blood flow images obtained from a patient). Instead, using the model-based approach of the present invention, different influences are incorporated to allow the prediction of the wide range of observations and features that can be encountered in diagnostic acquisitions. The approach of the first embodiment of the present invention offers the advantage of a well-defined possibility to include all a-priori knowledge on the observed process into the analysis over prior art computational feature analysis.

The further embodiments focus on the beneficial usage of extracted flow information for visualization and the presentation to observers in an easily accessible way. Different information and phenomena are either extracted and enhanced or filtered out and based on any deviations from predictions are brought to the attention of the physician/interventionalist such that further visualization of microflow phenomena (more detailed visualizations of identified anomalous flows) can be accomplished and visually compared by the physician/interventionalist with expected values.

In a second embodiment, contrast agent propagation contained in a sequence of diagnostic images is compared to modeled physiologic flow patterns that are matched to the observed sequence. The visualization and quantification of respective residual deviations is used to first identify anomalous flows and then to perform detailed analysis, such as comparison of the parameters extracted to distributions of expected values in the target vascular structures.

In a third embodiment, adaptive signal pre-processing (filtering) is applied during a filtering step to account for a specific patient's blood flow velocity, total blood flow, and other relevant flow parameter. An alternative includes adaptive filtering that depends on the replay speed in slow-motion replays.

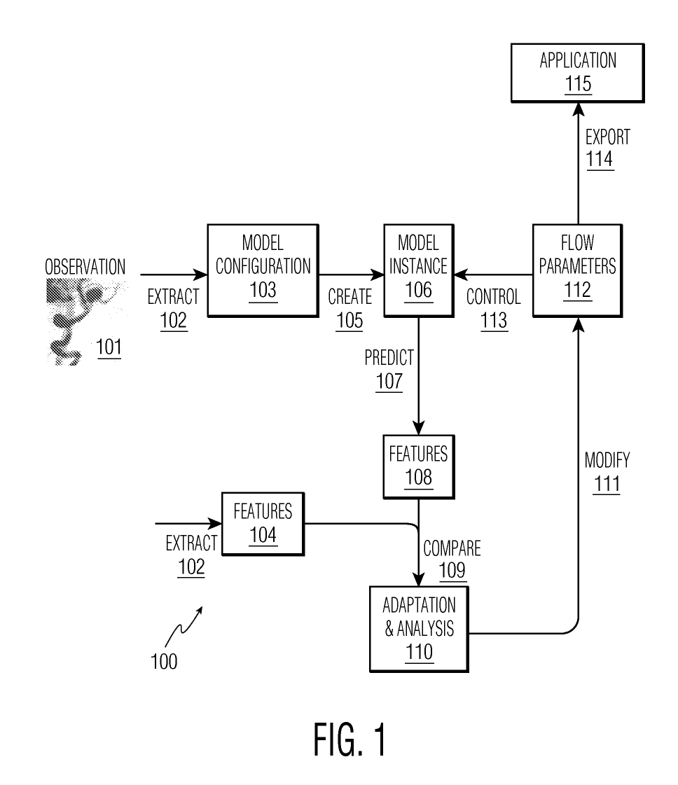

FIG. 1 illustrates a model based flow analysis workflow of the present invention and illustrates the use of extracted features to particularize a model and includes error measurement and correction of the resulting model for a specific patient;

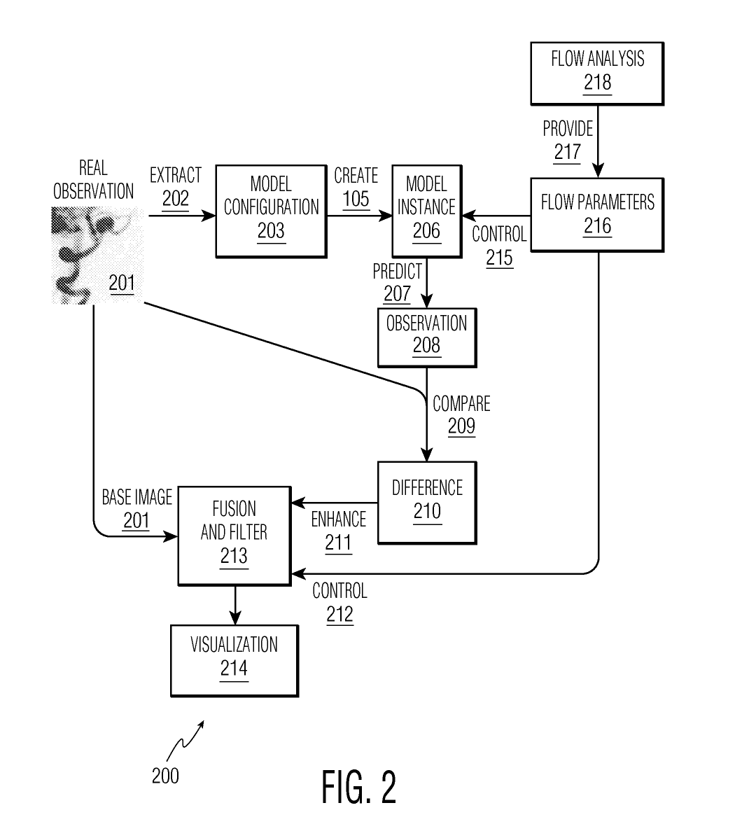

FIG. 2 illustrates the scheme for visualization of flow phenomena by determining differences between model predictions and the original observation;

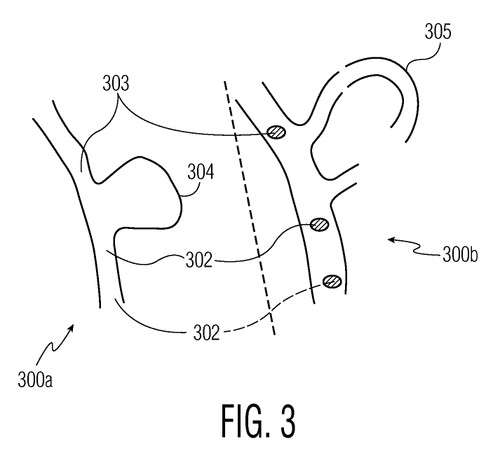

FIG. 3 illustrates an aneurysm with an observation point and an associated model according to the present invention;

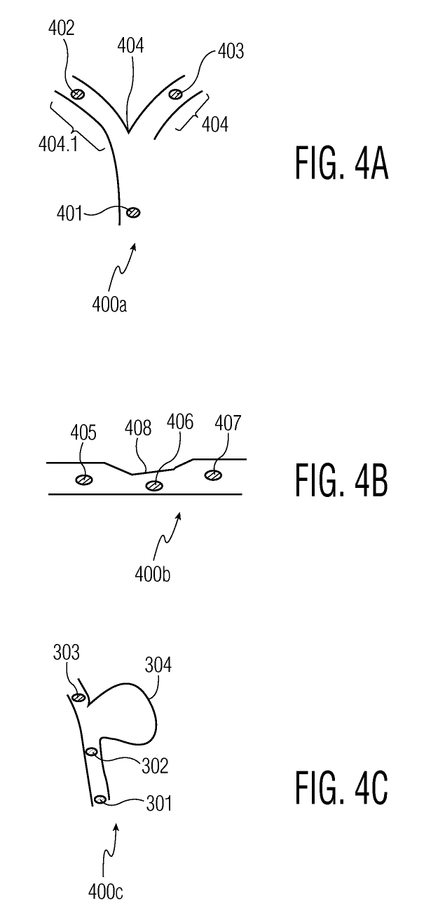

FIG. 4 illustrate examples of observation points associated with various vessel topologies;

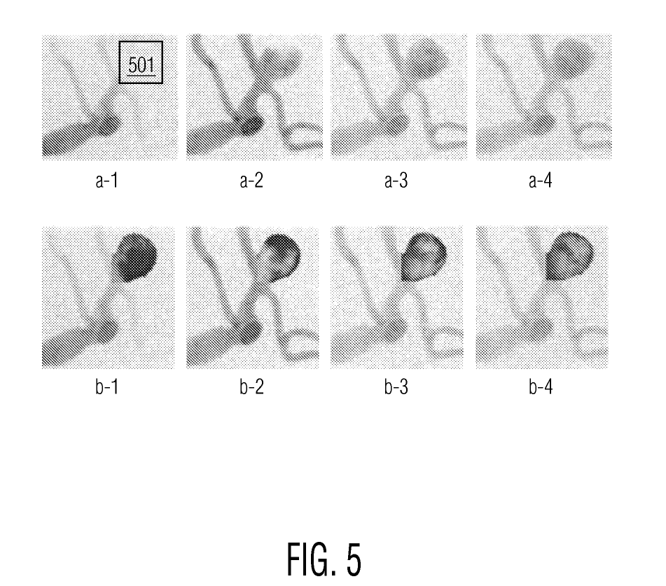

FIG. 5 illustrates an example of diagnostic images of blood vessel segments where the flow of contrast agent is observed in an aneurysm (original frames from the acquisition a) and processed images that visualize the microflow in this anomaly (b);

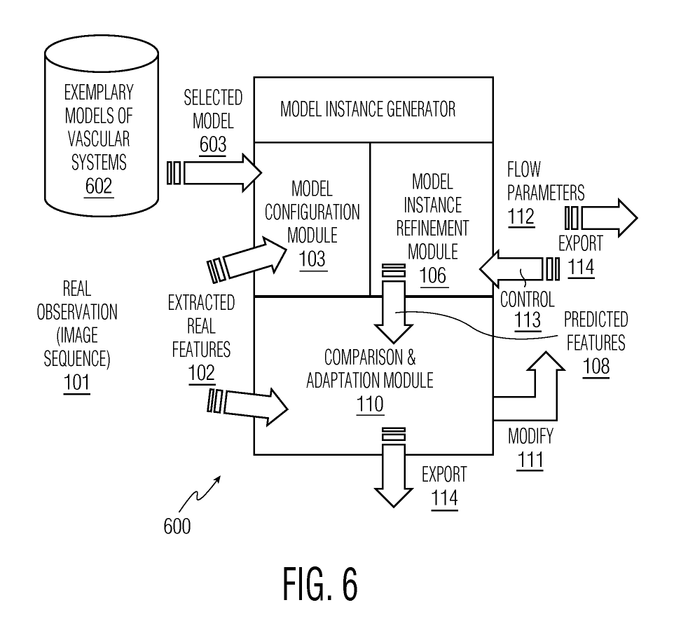

FIG. 6 illustrates an apparatus that implements the model based flow analysis of a first embodiment;

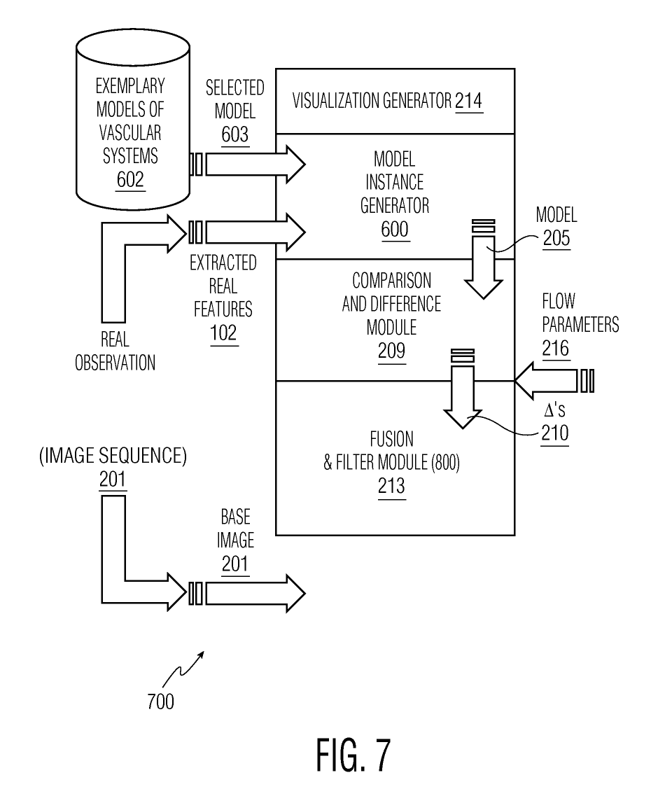

FIG. 7 illustrates an apparatus that implements the scheme for visualization of a second embodiment;

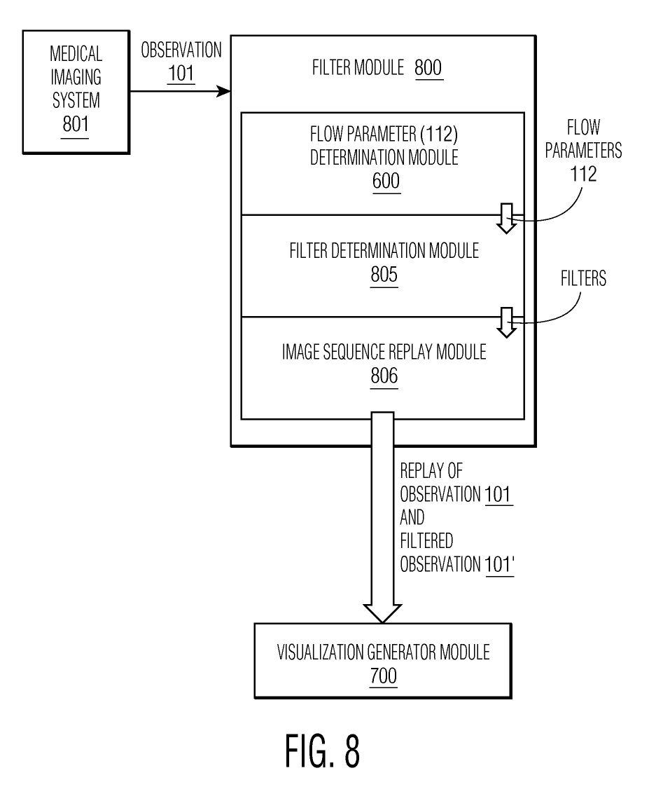

FIG. 8 illustrates an apparatus that implements filtering of images of a dynamic observation; and

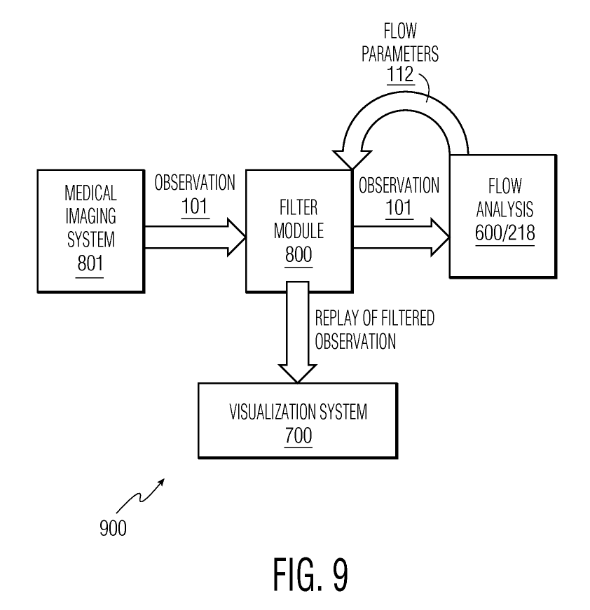

FIG. 9 illustrates a system for capturing a dynamic observation by an imaging modality, filtering the images according to the third embodiment of the present invention, applying the flow analysis of a first embodiment of the present invention to the filtered dynamic observation and visualizing a replay of the filtered and modeled dynamic observation with a second embodiment of the present invention.

It is to be understood by persons of ordinary skill in the art that the following descriptions are provided for purposes of illustration and not for limitation. An artisan understands that there are many variations that lie within the spirit of the invention and the scope of the appended claims. Unnecessary detail of known functions and structure may be omitted from the current descriptions so as not to obscure the present invention. Examples are for expository purposes only and are not intended as limitations on the scope of the invention.

In a first embodiment, the system, apparatus and method of the present invention provide an exemplary set of mathematical flow models covering the important vessel configurations and pathologies of interest to a physician/interventionalist and provide a manual or automatic selection technique of an appropriate model for a case under consideration. Each model comprises a parameter set that covers a set of specific flow parameters of a vessel topology or pathology. The aim of the model-based analysis of a preferred embodiment is to optimize this set and provide the parameters to the user when a model gives a prediction that is as similar as possible to an observation. Thus, the optimized model parameters comprise the clinically relevant information for diagnosis and outcome control for a vessel structure under consideration. In an alternative preferred embodiment, complex vessel systems can be analyzed by connecting several tailored models. Model selection depends on the vessel topology depicted in a sequence of at least one image and can either be performed manually or automatically.

Referring now to FIG. 1, in a preferred first embodiment, the present invention incorporates a priori knowledge of blood flow based on fluid dynamics of observed features to determine an appropriate flow model that is adapted to the real physiological process represented by an observation 101 consisting of a sequence of diagnostic image data that shows the advance of contrast agent in a vascular system. However, due to the different behavior of flow in different vessel topologies and pathologies, a tailored model for each vessel structure of interest is required. The present invention specifies an exemplary set of mathematical flow models covering important vessel topologies and pathologies of interest, and provides a selection technique for an appropriate model for each case under consideration. Possible further prediction models for other vascular subsystems include a tumor feed, an arterio-venous malformation, etc., but are examples only, and are not meant as a limitation of the method.

In a preferred embodiment, each model comprises a parameter set that spans the specific flow parameters of at least one of a vessel configuration and a vessel pathology. The present invention optimizes model parameters to reflect the clinically relevant information for diagnosis and outcome control for the vessel structure under consideration.

In an alternative preferred embodiment it is possible to connect several tailored models for analysis of a particular complex vessel system configuration. The resulting case-specific flow models and their selection enable blood flow assessment for any physiologically relevant structure, which is a prerequisite for such an analysis to be applicable to all different vascular configurations that can be observed in a patient. The model selection procedure of the present invention employs a vessel topology depicted in diagnostic imaging, i.e., a sequence of images.

For the model-based flow analysis of human blood flow, the main problems that can now be dealt with are the pulsatile nature of blood flow, all non-Newtonian fluid properties of blood with strong inter- and intra-patient variabilities and the influence of the contrast agent injection itself.

Thus, the model-based flow analysis paradigm provided by the system, apparatus, and method of the present invention incorporates required features into an algorithmic framework that allows its use for the analysis of clinical observations captured as a sequence of images. It is assumed in this model-based analysis paradigm that model parameters are valid and explain a real-world observation such that a plausible model prediction using these parameters results in features that have been observed previously.

A preferred embodiment of a method for the model-based flow analysis is illustrated in FIG. 1. The observed data 101 in the acquisition now provides two inputs 102 to the analysis framework. Representative features are extracted 104 that contain all required information of the flow process. Additionally, boundary conditions for the model are extracted to configure the model 103. In this context, boundary conditions are properties of the vasculature that need to be known for the later feature prediction 107 but are independent of the flow itself. In a preferred embodiment, the configuration of a model of vasculature contains all characteristic geometric properties that can be determined from an analyzed angiogram or that are available from other imaging modalities.

The model instance 106 predicts 107 features 108 dependent on flow properties when configured with boundary conditions. An adaptation loop 110-113 modifies flow properties until the predicted features 108 match, within a pre-determined tolerance, the extracted features 104 from the observation 101.

Once created, an adapted model instance 106 is available that can now predict features when controlled by flow parameters. This prediction is the characteristic step of the model-based analysis of the present invention because here, all available a-priori knowledge is included in the process. The comparison of features 104 extracted 102 from an observation 101 and the predicted 107 features 108 gives a measure of deviation or prediction error for the model. Relevant flow parameters are selected depending on the target application and form a search space. A suitable optimization algorithm is applied to adapt 110 these flow parameters 112 to reduce and finally minimize the prediction error. According to the model-based paradigm of a preferred embodiment of the present invention, those parameters that minimize the residual error between observation and model prediction are the result of the analysis and can be provided 114 to an application 115.

The quality of these results then depends on the validity and plausibility of the prediction and configuration of the model. In a preferred embodiment, these two essential properties are tuned for each application without the need to modify the analysis framework itself.

Model-based analysis determines a configured instance of a model that is able to predict and, therefore, explain an observation using plausible a-priori knowledge to deal with complex observations. In the creation of such a model-based analysis, in a preferred embodiment, every effect that should be represented in the analysis is included in the prediction 107 of features 108.

An example of a method 100 according to a first embodiment is given for interventional x-ray but is not meant to limit the method to this modality:
1. Imaging of bolus injection under X-ray surveillance: A contrast agent is injected into a vessel of interest in order to make a blood flow visible in a sequence of at least two x-ray images. For this purpose, specific pre-determined injection protocols are used.
2. Identifying vessel structures and selecting a flow model: An opaque mask of a vessel tree is calculated by performing maximum/minimum operations on a sequence of at least two x-ray images. Subsequently, the physician/interventionalist selects an appropriate model from a provided standard set of models by a visual inspection of the opaque vessel tree.
   Each flow model provided by a first embodiment describes the transport of contrast agent for a specific configuration. Via the flow models, a prediction is made of the time intensity curves considered at features here, i.e. the concentration of contrast agent varying over time at a pre-determined set of observation points. Each model includes a model-specific parameter set that covers at least one specific feature of a vessel topology or pathology and requires a different number of at least one pre-defined observation point. As a result, specific blood flow related parameters are extracted for the vessel configuration of interest.
   The set of flow models comprises, but is not limited to, models for stenosis, aneurysm and bifurcation. An example of the extraction of clinically relevant information from a custom-built flow model is stenosis grading. In prior art clinical routine, stenosis grading is performed by measuring the pressure decrease over a stenosis by utilizing a pressure wire. This procedure can be mimicked by a blood flow measurement under x-ray surveillance. A procedure measures the pulsatile volumetric blood flow and the pulsatile velocity at any observation point in a non-branched vessel from a sequence of contrasted x-ray images or acquired by a similar suitable modality. By means of this approach the velocity v(t) can be calculated for several observation points over the stenosis, see FIG. 4b. Note that the volumetric blood flow Q(t) is identical for each observation point. By exploiting v(t) and Q(t), the effective radius R of the stenosis at each observation point is subsequently calculated by

Q(t)=v(t)πR². (1)
The relationship between pressure decrease Δp, effective radius R and volumetric blood flow Q(t) is known in the art. As a result, a calculation of the pressure decrease over the stenosis can be performed.
   In an alternative embodiment, pressure decrease measurement is performed using a velocity-based stenosis grading. Here, the degree of the stenosis is calculated by

$$( 1 - \frac{{v}_{1}}{{v}_{2}} ) 100 \%$$

where v₁ is the velocity at observation point 1 and v₂ is the velocity at observation point 2.
For the above analysis, the flow model is created to predict the transport of contrast agent through tubular structures between observation points. This prediction can preferably take into account all mechanisms of the blood and contrast transport, mainly pulsatile dispersion, diffusion, and the varying blood velocity over a vessel cross section.
   Another example of the extraction of clinically relevant information from custom-built flow models is the assessment of aneurysms. Here, the fraction of volumetric blood flow taking the detour through the aneurysm is of interest to the physician. The fraction of blood flow from the parenting vessel that flows through the aneurysm volume is required to determine the residual time of blood in the aneurysm, which is considered a relevant parameter for treatment decision and outcome control.
   First, the overall volumetric blood flow is determined by simulating the contrast agent transport between the observation points 301-302 in a feed, see 300a of FIG. 3a. Subsequently, the fraction taking a detour through the aneurysm 304 and the fraction passing by the aneurysm without entering is calculated. For this purpose, the contrast agent transport from a second observation point 302 to the third observation point 303 is simulated by using the model depicted in FIG. 3 element 300b. This underlying model consists of two tubular structures connecting the two observation points 305 306. The first tubular structure 306 models the original physiologic connection of the observation points, whereas the second tubular structure 305 models the detour the contrast agent takes in the aneurysm 304. The length and radius of each tubular structure are parameters in the optimization routine and the contrast agent dynamics in each of the modeled tubes are preferably modeled as tubular vessels as described above. As further embodiment, to predict the concentration and amount of contrast agent within an aneurysm sac, the aneurysm is modeled as a fluid volume with homogenous contrast concentration inside, which is predicted according to the amount of contrast agent that flows in via the observation point 302.
   Another example for the extraction of clinically relevant information from custom-built flow models is the assessment of a bifurcation (see 404 of FIG. 4a) using the ratio of volumetric blood flows in the branches 404.1 404.2. For this purpose the contrast agent transport from an observation point in the feed 401 to observation points 402 403 in each branch of the drain is simulated preferably using the model for contrast transport in tubular structures given above. One of the parameters of this simulation is the fraction of flow into each of the branches 402 403. The ratio of these scaling factors indicates the ratio of volumetric blood flow in the drains (branches) 402 403.
3. Extracting time intensity curves (TICs) at relevant observation points: For all observation points given above, in a preferred embodiment the local concentration of contrast agent is determined taking an average of the intensity of the contrast agent in a pre-specified area around an observation point in a vessel in order to reduce the influence of noise. The number and location of observation points depends on the present vessel topology or pathology and therefore on the flow model.
4. Optimizing model parameters: The flow model provides a prediction of features, preferably of the time intensity curve and concentration of contrast agent along a vessel at each observation point. In an optimization procedure of a preferred embodiment, the predicted and the observed TICs are compared 109 and the model parameters are adjusted such that the error between the measured time intensity curve and the model prediction is minimized. The output parameters then provide important diagnostic values for the assessment of a disease. In the case of a bifurcation, component 404 of FIG. 400a, in a preferred embodiment this is the ratio of volumetric blood flow in the branches 404.1 404.2 as indicated above, whereas for a stenosis, component 408 of FIG. 400b, this is the pressure decline over the stenosis 408. In the case of an aneurysm, the fraction of blood flow from the parenting vessel that flow through the aneurysm itself is the major parameter.
5. Displaying relevant output parameters: In a preferred embodiment, flow parameters 112 are displayed to the physician/interventionalist in an appropriate way. In an alternative embodiment, results are passed on to applications 115 that process the results from flow analysis.

Referring now to FIG. 6, an apparatus 600 that implements the second embodiment is illustrated, comprising a model instance generator that controls a model configuration module in the selection and initial configuration (based on extracted real features) of an appropriate model from a database 602 of exemplary models of all possible vascular systems of interest. The model instance refinement module 106 executes the model to obtain predicted features 108 which are then compared to the extracted real features and values of flow parameters associated with the selected model are adapted by a comparison and adaptation module 110. The adapted flow parameters are used to refine the model instance by the model instance refinement module 106 and the process of prediction, comparison, adaptation and refinement is repeated until the differences between the real and predicted features fall within at least one pre-determined tolerance. The finally determined flow parameters from this iterative process are exported 114 to other system/applications for use thereby, e.g., for use in a second embodiment that is described below.

Use of Models for Flow Visualization

A second embodiment, see FIG. 2, is a model-based visualization mechanism in which different information and phenomena are one of extracted/enhanced, and filtered out. The decision to make an enhancement or perform a filter process is made during the prediction step 207.

In the model-based visualization framework of the second embodiment, selected parts of a real observation 201 are explained by a configured model 206 and can be either suppressed or specially handled. The difference 210 between a predicted observation 208 and a real observation 201 contains all information filtered by the a-priori knowledge available in the model prediction step 207.

For the model-based visualization scheme of the second embodiment, the model instance is fixed. Boundary conditions on vascular geometry are again extracted 202 from the real observation. For a flow analysis of contrasted angiograms, this prediction includes the local amount of contrast agent in vascular subsystems of interest. Furthermore, dynamic flow parameters are fixed as well. These are usually provided by a prior flow analysis. The model instance 206 provides increased prediction abilities in this second embodiment. The filtering or selection of relevant contents of the visualization is obtained by a subtraction from the true observation 201 of the model-predicted observation 208. This difference contains all flow phenomena that have not been explained by the model instance itself 206. Advantageously, the model instance 206 is created such that it can explain and predict physiologic flow phenomena. The difference 210 of the observation predicted 208 by the model instance 206 and the real observation 201 then contains all deviations from normal physiologic flow. A fusion 213 of original observation 201 with residual differences of the physiologic prediction is then used in the second embodiment to enhance, e.g., color-code, all pathologic or inexplicable flow phenomena.

The enhanced visualization 214 of these differences in the second embodiment is a significant advance over the prior art because, usually, all microflow effects are obscured by the contrast agent in physiologic flow patterns and, therefore, the presence of the contrast agent strongly attenuates the vascular structures of interest. The fusion and image filter 213 parameters that are applied in a second preferred embodiment of such a visualization 214 are beneficially taken from the flow parameters themselves. In particular, the expected temporal dynamics of the contrast agent are used to control 205 noise reduction filters in this fusion step 202, in a third embodiment disclosed below.

Referring now to FIG. 7, an apparatus 700 that implements the second embodiment is illustrated, comprising a model instance generator 600 according to a first embodiment that is used by a comparison and difference module 209 to obtain predicted observations and compare the predicted observation to a base image (a real observation 201) and derive differences therebetween 210 which differences are then visualized with respect to the base image (the real observation 201) by a fusion & filter module 213, the filter being an implementation of a third embodiment 800.

In an example of the second embodiment, see FIG. 5, an aneurysm sac is modeled as one homogenously mixed chamber containing contrast agent in exchange with the parenting vessel stream. Referring now to FIGS. 5a1-a4, frames from a diagnostic acquisition show the arrival of contrast agent in the aneurysm sac. The geometry of this aneurysm sac is extracted from an opaque mask of the vasculature in the flow sequence when diagnostic x-ray angiograms are taken as input (see item 2, above). In a user-selected ROI (shown as a rectangle 501 in FIG. 5 a-1), the maximal attenuation stored in the trace subtract image is threshold-segmented to determine the endovascular lumen in projection. As a result, a map contains the endovascular lumen and the maximal contrast agent concentration (representative for the local thickness) of the aneurysm. The total amount of contrast agent in the aneurysm is extracted. Scaling the aneurysm map with this total amount is used in model prediction to remove the influence of the total attenuation from the visualization. The subtraction of this modeled contrast agent concentration from the observation itself reveals microflow in the aneurysm independent of the momentary attenuation within (FIGS. 5 b1-b4).

An alternative second embodiment introduces color (not shown) that allows enhancement of the appearance of greylevel angiograms without modification of the original diagnostic information and greatly improves the attention-getting quality of the colored angiogram as well as its diagnostic usefulness. For such a color visualization, in the diagnostic observation I(x,y,t), the greylevels I correspond to the local concentration of contrast agent at a position (x,y) at time instance and, therefore, image frame t. The model prediction provides an image sequence P(x,y,t) that contains all the predicted contrast agent concentrations P provided by the model at positions (x,y) and time t. The difference D (x,y,t) of these two image sequences therefore contains all non-explained contrast agent variations. In a preferred visualization, the original acquisition I is used to determine the local intensity of a visualization and the local difference D is used to select the coloration, preferably without a modification of the intensity itself.

In yet a further alternative second embodiment, a synthetic view of an imaged vascular structure is created. For this, the extracted geometry is displayed as a sketch of the vasculature. Color schemes can be used for each vessel segment with a selected flow parameter. The volume flow or the degree of pulsatility is a possible local parameter in the flow tree that can be visualized in such an overview sketch. In particular, unexpectedly high or low values can be indicated by a classification of extracted data in statistical distributions obtained from physiologic vasculatures. Such a colored sketch can either serve as an overview for the state of subtrees in a complex vasculature or as a function of the runlength in a pathologically affected vessel. In contrast to the first alternative embodiments, here a new and synthetic display is created from the model and extracted parameters.

Use of Flow and Replay Parameters for Filtering

Image filtering to reduce noise and artifacts is regularly applied to all medical image data. However, filtering with improper technical parameters can obscure important observations or even create artifact structures that are visible to the observer's eye but have never been in the acquired data. A third embodiment addresses these issues by using information concerning individual patient blood flow speeds (that vary over time due to heart beat) to tune filters such that the images contain as little noise as possible but on the other hand always show contrast agent bolus motion without blurring (which is one of the most frequent image quality degradations that a filter can introduce when not properly tuned). In the third embodiment image (pre-) processing and its parameters are dependent on an estimated flow velocity, total blood flow, or any other relevant flow parameter of a patient's anatomy depicted in a sequence of at least one image, e.g., x-ray.

An example of the third embodiment is the reduction of image noise by temporal filtering. Here, the strength of temporal filtering depends on the blood flow velocity. The filtering strength can vary with time and location since the flow velocity is time-dependent due to pulsatility and the flow velocity strongly varies in different vascular systems that can be observed.

A preferred embodiment of a method according to the third embodiment comprises the steps of:
1. Injecting a contrast bolus by the interventional radiologist into the vessel of interest.
2. Measuring flow speed from the acquired x-ray sequence with the use of well known videodensitometric techniques and the technique of the first embodiment. As result, characteristic flow parameters like the flow velocity or the total flow volume are obtained
3. Temporally filtering with adaptive filter scale that avoids blurring by allowing the bolus only to cover the maximal distance d in the weighted and averaged frames. Introducing the flow velocity v (estimated as before), the optimal time period Δt covered by the weighted and averaged frames is Δt=d/v. Since additionally the frame rate f of the sequence is known, the standard deviation σ of a Gaussian lowpass filter used for temporal filters can be calculated by

$$σ = \frac{d * f}{v} .$$

Since the flow velocity v(t,x) is a function of time and location, the standard deviation σ that reflects the strength of temporal filtering can be calculated for each individual time instant and pixel (of the vessel) individually (whereas outside the vessel an appropriate strong standard deviation σ can be chosen). However, if such a local strength of noise suppression is used, it might result in the visual impression of a flickering sequence. To solve this problem, 2 possibilities exist:
   a. Instead of using a local strength of noise suppression, a global σ a is used. To obtain the appropriate global σ, the maximal flow velocity max_{Λx,t}(v(t,x)) of the image sequence has to be known. The flow speed is measured at least over a full heartbeat. During that time either no temporal filtering or preferentially a time adaptive temporal filtering that uses the maximal measured velocity so far is performed. Preferably, the first embodiment is used to determine the flow velocity and its change over the cardiac cycle.
   b. If a local strength of noise suppression is used, an appropriate regularization over the image and over time is performed.
4. Replaying of x-ray images instantaneously after temporal filtering or in a slow-motion replay after image acquisition. An additional latency to that already required for the standard pre-processing is introduced for the instantaneous replay by the additional blood flow assessment. However, since the image quality is more important in a slow-motion replay as the human eye does not average the screen sequence in this case, the real time requirement is not of great importance.

In an alternative third embodiment, the strength of the applied noise filters further depends on the replay speed that a user has selected when a slow motion replay is offered by the apparatus. The strength of temporal filters can be increased for faster replays giving a noise-free visualization whereas for lower replay speeds, the temporal filter strength is reduced to avoid a respective blurring that becomes more and more obvious when individual frames are seen in slow motion.

Referring now to FIG. 8, an apparatus for a filter module 800 is illustrated. Flow parameters 112 are determined using the first embodiment and a filter determination module 805 selects, adjusts and applies filters in according with at least one of flow speed (a flow parameter 112) and replay speed. The observation is replayed by an image sequence replay module 806 that uses a second embodiment of the present invention to visualize the transport of a contrast agent in an observation contained in a real observation as compared with a filtered observation.

Referring now to FIG. 9, a system comprising a medical imaging system 801 that provides a real diagnostic observation 101 to a filter module 800 that applies filters selected thereby (using flow parameters 112 resulting from an application of a first embodiment) to a replay of the real and possibly modeled flow (predicted flow) resulting from a flow analysis 600 which filtered replay is then visualized by a third embodiment 700.

While the preferred embodiments of the present invention have been illustrated and described, it will be understood by those skilled in the art that the system, apparatus and methods as described herein are illustrative and various changes and modifications may be made and equivalents may be substituted for elements thereof without departing from the true scope of the present invention. In addition, many modifications may be made to adapt the teachings of the present invention to a particular situation without departing from its central scope. Therefore, it is intended that the present invention not be limited to the particular embodiments disclosed as the best mode contemplated for carrying out the present invention, but that the present invention include all embodiments falling within the scope of the claims appended hereto.

1. A method (100) for analyzing blood flow in a vascular system from a diagnostic observation thereof, comprising the steps of:
providing a database (602) of at least one exemplary blood flow model of a vascular system, the at least one model having an associated parameter set of the most relevant blood flow parameters of the modeled vascular system;
providing a diagnostic observation (101) as a sequence of at least two images that show the advance of contrast agent in the vascular system;
extracting a set of extracted quantitative blood flow features (102) of the vascular system using the parameter set of the model, from the provided diagnostic observation (101);
selecting (103) and linking at least one blood flow model (603) for the observed vascular system from the database (602) such that predicted flow blood features (108) predicted by the model match the extracted blood flow features (102) according to a pre-determined matching function of the associated set of blood flow parameters; and
outputting the model and the associated parameter set of blood flow parameters.

2. The method (100) of claim 1, further comprising the step of the selected model predicting at least one of the diagnostic observation (101) and the set of quantitative features extracted (102) from the diagnostic observation (101).

3. The method (100) of claim 2, wherein the pre-determined matching function comprises the step of systematically varying the values of blood flow parameters of the selected model until the predicted features and corresponding extracted features each differ from one another by less than a pre-specified tolerance.

4. The method (100) of claim 3, wherein the systematically varying is executing numerical optimization routines.

5. The method (100) of claim 4, wherein a local concentration of a contrast agent at an observation point is both one of the extracted features (102) and one of the predicted features (108).

6. The method (100) of claim 5, further comprising the step of:
injecting the contrast agent into a blood vessel of the vascular system; and
wherein, the diagnostic observation (101) is a diagnostic x-ray obtained by performing the step of taking a series of at least two x-ray images of the vascular system after the injection step.

7. The method (100) of claim 8, further comprising the step of determining a geometry of the vascular system from the diagnostic x-ray taken thereof.

8. The method (100) of claim 8, further comprising the step of presenting the flow parameters to the user.

9. The method of claim 1, wherein the exemplary model set (602) includes a model that describes the flow of contrast agent through tubular structures with transport mechanisms selected from the group consisting of dispersion, diffusion, convection, varying velocities over a vessel cross-section, and varying velocity over a heart cycle.

10. The method (100) of claim 1, wherein:
the database (602) includes a model of a stenosis; and
and the set of extracted features (102) includes a grade of the stenosis.

11. The method (100) of claim 1, wherein:
the database (602) includes a model comprising at least one parenting tube and at least two branching tubes forming a bifurcation thereof; and
the set of extracted features (102) includes a flow fraction into the at least two branching tubes.

12. The method (100) of claim 1, wherein:
the database (602) includes a model of an aneurysm sac of a vessel having a parenting vessel; and
the set of extracted features (102) includes a fraction of a flow of the parenting vessel that flows through the aneurysm.

13. The method (100) of claim 13, wherein the aneurysm sac comprises two parallel tubular vessels, one for the parenting vessel and one replacing the aneurysm itself.

14. The method (100) of claim 14, wherein the aneurysm sac comprises a fluid chamber with homogenous contrast concentration.

15. An apparatus (600) for analyzing blood flow in an observed vascular system from a diagnostic observation (101) thereof, comprising:
a database (602) of exemplary models of blood flow in vascular systems, each model having an associated set of blood flow parameters most relevant to the modeled vascular system; and
a model instance generator (600) to control creation of an instance of at least one exemplary model selected from the database (602) based on extracted features (102) of the observed vascular system and linked such that predicted blood flow features (108) predicted by the at least one model match extracted blood flow features (102) according to a pre-determined matching function of the associated set of blood flow parameters.

16. A method (200) for visualization of blood flow in a vascular system from a diagnostic observation thereof, comprising the steps of:
determining a blood flow model and blood flow parameters thereof by performing the method of claim 1 such that the model predicts the observation based on the flow parameters; and
providing a visualization apparatus (700) to visualize the blood flow of the model based on the flow parameters.

17. The method (200) of claim 16, further comprising the steps of:
generating a predicted observation using the determined blood flow model; and
visualizing with the provided visualization apparatus the observation and differences between the observation and the predicted observation.

18. The method (200) of claim 16, further comprising the steps of:
generating a predicted observation using the determined blood flow model;
visualizing with the provided visualization apparatus the observation; and
enhancing the visualized observation with a function of differences between the observation and the predicted observation.

19. The method (200) of claim 18 wherein the function is a color overlay created from the differences.

20. The method (200) of claim 19, wherein the predicted observation is a concentration of contrast agent in a tubular vessel when the concentration at an inflow into the segment is observed over time as the diagnostic observation.

21. The method (200) of claim 20 wherein the generating further comprises the step of including contrast transport effects of transport mechanisms selected from the group consisting of dispersion, diffusion, convection, varying velocities over a vessel cross-section, and varying velocity over a heart cycle.

22. The method (200) of claim 20, wherein:
the extracted blood flow features (102) include an amount of a contrast agent in the vascular system or a part thereof; and
the generating step further comprises the step of assuming a homogenous concentration of the contrast agent in vascular system or a part thereof

23. The method (200) of claim 22, wherein:
the extracted blood flow features (102) include a geometry of the vascular system or part thereof; and
the generating step further comprises the step of including information on the geometry of the vascular system or part thereof.

24. The method (200) of claim 22, wherein the generating step further comprises the step of including information on the geometry of the vascular system or part thereof obtained from an alternative modality than the diagnostic observation.

25. An apparatus (700) for visualization of blood flow in a vascular system from a diagnostic observation thereof:
a database (602) of exemplary models of blood flow in vascular systems, each model having an associated set of blood flow parameters most relevant to the modeled vascular system;
an apparatus (600) according to claim 15 to create a model instance of an exemplary model of the database (602) for analyzing blood flow in an observed vascular system from the diagnostic observation (101); and
a visualization generator (214) to visualize a base image (201) of the diagnostic observation for visual comparison with a predicted observation (208) predicted by the model instance.

26. The apparatus (700) of claim 25, wherein the visualization generator (214) is further configured to perform the method of claim 24.

27. A method for filtering a dynamic diagnostic observation sequence showing the advance of a contrast agent in a vascular system therein, comprising the steps of:
locally determining the strength of a temporal filter based on at least one criteria selected from the group consisting of:
a local blood velocity of the diagnostic observation, and
a blur due to filtering that only covers a pre-defined distance that the contrast agent can pass over in an observation time defined by a filter scale;
determining a global filter strength from the locally determined filter strengths by minimum comparison; and
applying a pre-determined number of filters selected from the group consisting of a temporal filter and a global filter to the observation.

28. The method of claim 27, further comprising the step of prior to the applying step, regularizing the strength of each temporal filter.

29. The method of claim 28, wherein the regularizing step is implemented by spatial and temporal lowpass of the filter strengths.

30. The method of claim 29, further comprising the step of simultaneously with the applying step, performing the method of claim 24 to visualize the advance of the contrast agent in the vascular system.

31. A method for filtering a dynamic diagnostic observation sequence to visualize the advance of a contrast agent in a vascular system therein, comprising the steps of:
providing a replay speed that is adjustable;
adjusting the replay speed;
selecting noise filters based on the selected replay speed; and
simultaneously performing the steps of:
a. applying the selected noise filters, and
b. performing the method of claim 25 to visualize the advance of the contrast agent in the vascular system.

32. The method of claim 31, further comprising the steps of:
applying a strong temporal filter when the replay speed exceeds a pre-determined strong threshold; and
applying one of a weak and no temporal filter when the replay speed falls below a pre-determined weak threshold.

33. The method of claim 31, wherein:
the providing step further comprises providing a continuous rate of change of the replay speed; and
and when the rate of change of the replay speed is continuous, the selecting step further only comprises a continuous change of the temporal filter strength.

34. An apparatus (800) for filtering a dynamic diagnostic observation sequence of contrast agent advance in a vascular system, comprising:
a flow parameter determination module configured as in claim 15, to determine flow parameters (112) of the observation;
a filter determination module 805 configured to perform the method of claim 30 to determine a pre-determined number of filters to be applied to the observation sequence;
an image sequence replay module (806) to determine a replay speed and a filter strength of the filters based on the replay speed using the method of claim 33 and to output a filtered replay of the observation sequence (101′); and
a visualization generation module configured as in claim 27 to accept and visualize the filtered replay of the observation sequence (101′) output by the image sequence replay module (806).

35. A system (900) for filtering replaying and visualizing a dynamic observation sequence (101), comprising:
a filter module (800) to
determine at least one filter from flow parameters (112) of the dynamic observation sequence and
replay at a determined speed the filtered dynamic observation sequence (101);
a flow analysis module operably connected to the filter module (800) to determine flow parameters (112) of the dynamic observation sequence and provide said determined flow parameters to the flow analysis module (800); and
a visualization system (700) operably connected to the filter module to visualize at least one of a replay of the filtered sequence and a replay of the observation.
